(19) United States
(12) Patent Application Publication
(10) Pub. No.: US 2021/0170192 A1
(43) Pub. Date: Jun. 10, 2021

(54) PHOTOTHERAPY SYSTEM

(71) Applicant: SUZHOU INSTITUTE OF BIOMEDICAL ENGINEERING AND TECHNOLOGY, CHINESE ACADEMY OF SCIENCES, Suzhou (CN)

(72) Inventors: Peipei WANG, Suzhou (CN); Daxi XIONG, Suzhou (CN)

(21) Appl. No.: 17/073,350

(22) Filed: Oct. 18, 2020

Related U.S. Application Data
(63) Continuation of application No. PCT/CN2018/091364, filed on Jun. 14, 2018.

(30) Foreign Application Priority Data
Apr. 19, 2018 (CN) 201810352866.7
Apr. 19, 2018 (CN) 201810352932.0
Apr. 19, 2018 (CN) 201820559229.2
Apr. 19, 2018 (CN) 201820559230.5

(51) Int. Cl. A61N 5/06

(52) U.S. Cl. CPC A61N 5/0616; A61N 2005/063; A61N 2005/005; A61N 2005/0645; A61N 2005/0644; A61N 2005/0652

(57) ABSTRACT

The object of the present application is to provide a phototherapy system, comprising a light source component, a light guide component and a phototherapy component, wherein the light source component can emit light of one or more specific wavelengths for illumination during phototherapy, the light source can be an LED light source; the light guide component is provided between the light source component and the phototherapy component, for guiding the light by the light source component, after undergoing beam shaping and contraction by the beam shaping module, to the phototherapy component. The present application improves the safety and applicability the product by separating the generation portion from the application portion of the light source, and provides a solution for unified treatment of a plurality of human body parts.

### TECHNICAL FIELD

The present application relates to the field of health care, in particular to a phototherapy system.

### BACKGROUND

A number of existing clinical medical experiments have shown that light irradiation with a specific spectrum can effectively treat a variety of diseases. The red light and infrared light act on the human body to mainly improve local blood circulation, promote swelling regression and analgesia, reduce muscle tone, and relieve muscle spasm and dry exudative lesions; ultraviolet rays have anti-inflammatory, analgesic, anti-rickets effects, commonly used to treat skin suppurative inflammation and other dermatitis, pain syndrome, rachitis or osteomalacia; visible light is also widely used, for example, red light for promoting wound healing, yellow light for skin rejuvenation, blue light for treatment of neonatal jaundice, etc.

At present, there are many portable phototherapy systems on the market. The portable design has the advantages of being light, not affecting daily life, and being used and promoted in homes.

However, the existing portable phototherapy system generally uses a plurality of LED lamp beads arranged in an array or other ways to irradiate the skin surface. The LED lamp beads are in contact with or not far apart from the body surface to be treated, thus the thermal energy generated by the LED lamp beads may interfere with the effect of treatment, and the circuit of the LED lamp bead is also not far away from the body surface, and there is a potential safety risk of leakage.

### SUMMARY

An object of the present application is to provide a phototherapy system to solve the problems that the phototherapy systems in the prior art have poor treatment effect and potential safety hazards of electric leakage.

According to one aspect of the present application, a phototherapy system is provided, including a light source component, a phototherapy component and a light guide component arranged between the light source component and the phototherapy component, wherein,

the light source component comprises: a light source module and a power supply module electrically connected thereto, a heat dissipation module and a beam shaping module respectively connected with the light source module, and a light guide interface connected with the beam shaping module and aligned with a centre of the beam shaping module;

the light source component is connected with the light guide component via the light guide interface; and

the phototherapy component comprises a contactable photocosmetic device and/or a wearable phototherapy device;

wherein, the power supply module is used for supplying power for the light source module, the light source module is used for emitting emergent light, the beam shaping module is used for performing beam shaping and contracting to the emergent light, the light guide component is used for guiding the emergent light after the beam shaping and contracting to the phototherapy component and provide phototherapy to a user through the phototherapy component, and the heat dissipation module is used for exhausting thermal energy generated by the light source module.

Further, in the above phototherapy system, the wearable phototherapy device comprises: an optical fiber light-emitting unit and a wearable unit,

wherein, the optical fiber light-emitting unit has one end connected with the light guide component, and the other end connected with the wearable unit; and

the light guide component is used for guiding the emergent light after the beam shaping and contracting to the optical fiber light-emitting unit, to provide phototherapy to a user wearing the wearable unit.

Further, in the above phototherapy system, the optical fiber light-emitting unit is a whole piece of optical fiber fabric formed from a first optical fiber filament woven together,

wherein, the light guide component transmit the emergent light to the first optical fiber filament, to allow the emergent light in the whole woven piece of optical fiber fabric to form a surface light source.

Further, in the above phototherapy system, the phototherapy system comprises a light source component, one or more phototherapy components and the light guide component corresponding thereto.

Further, in the above phototherapy system, the light source component comprises: a power supply module, a heat dissipation module, one or more light source modules and the beam shaping module and the light guide interface corresponding thereto, wherein,

each light source module corresponds to a group of the beam shaping module and a group of the light guide interface.

Further, in the above phototherapy system, two ends of the light guide component are respectively provided with an interface connected with the light source component and the wearable phototherapy device; wherein,

the interface on one end of the light guide component is connected with the light guide interface, and

the interface on the other end of the light guide component is connected with an interface on one end of the optical fiber light-emitting unit.

Further, in the above phototherapy system, the contactable photocosmetic device comprises an optical fiber layer, having a second optical fiber filament distributed therein and connected with the light guide component;

wherein, the light guide component is used for guiding the emergent light after the beam shaping and contracting to the optical fiber layer, to provide phototherapy to a user attached to the contactable photocosmetic device.

Further, in the above phototherapy system, the optical fiber layer forms a whole piece of optical fiber fabric in the form of the second optical fiber filament in a certain layout, or the optical fiber layer forms a whole piece of optical fiber fabric in the form of the second optical fiber filament which is interwoven in the optical fiber layer and is woven together;

wherein, the light guide component transmits the emergent light after the beam shaping and contracting to the second optical fiber filament, to allow the emergent light in the optical fiber fabric after the beam shaping and contracting, to form a surface light source.

Further, in the above phototherapy system, the contactable photocosmetic device further comprises a contact layer, which is fitted and connected with the optical fiber layer; or

the contactable photocosmetic device further comprises a contact layer and a reflective layer, wherein, the optical fiber layer has one surface fitted and connected with the contact layer, and another surface fitted and connected with the reflective layer.

Further, in the above phototherapy system, an edge of the optical fiber layer is provided with a docking interface, two ends of the light guide component are respectively provided with an interface connected with the light source component and the contactable photocosmetic device, wherein, the interface on one end of the light guide component is connected with the light guide interface, the interface on the other end of the light guide component is connected with the docking interface of the optical fiber layer; or

the edge of the optical fiber layer is provided with a docking interface, and only one end of the light guide component is provided with an interface fitted and connected with the docking interface, wherein, the docking interface secures the second optical fiber filament into a bundle of optical fiber via a hoop to form the interface of the light guide component.

Further, in the above phototherapy system, the beam shaping module comprises: a light guide post and a plano convex lens, wherein, the light guide post is respectively optically coupled with the light source module and the plano convex lens, and central lines of the light source module, the light guide post and the plano convex lens are along the same line, the light guide post is used for performing spectral mixing to the emergent light emitted by the light source module, the plano convex lens is used for compressing an emergence angle of the emergent light after the spectral mixing, to obtain emergent light after the beam shaping and contracting;

or,

the beam shaping module comprises a lens group, with one side thereof being in optical coupling connection with the light source module, and the other side thereof being in optical connection with the light guide interface, and being used for compressing the emergence angle of the emergent light emitted from the light source module.

Further, in the above phototherapy system, the heat dissipation module comprises a radiator, or a radiator and a fan,

wherein, the light source module is fixedly connected with a surface of the radiator of the heat dissipation module.

Further, in the above phototherapy system, the power supply module comprises: a control circuit and a direct constant current drive power supply; or

the power supply module comprises: a control circuit, a direct constant current drive power supply and a battery.

Further, in the above phototherapy system, the light source component is provided with a button, for switching wavelength and/or intensity of the emergent light, or adjusting a lighting mode.

Further, in the above phototherapy system, the phototherapy component is provided with two sets of corresponding fixation buckles, for fixing a treated site of the user during treatment process.

Further, in the above phototherapy system, the wearable phototherapy device is in at least any one of the following forms of:

wearable hats, wearable collars, wearable vests, wearable bellybands, wearable pants, wearable eye masks, wearable facial masks, wearable elbow protectors, wearable socks, wearable knee pads, wearable shoulder straps, wearable gloves and wearable belts;

the contactable photocosmetic device is in at least any one of the following forms of: contactable facial masks, contactable nasal masks and contactable eye masks.

Compared with the prior art, the phototherapy system in the present application includes a light source component, a phototherapy component, and a light guide component disposed between the light source component and the phototherapy component. The light guide component has good insulation, only guides light, but does not conduct heat, thus the power supply of the phototherapy system has high safety and is not liable to produce side effects. The light source component includes: a light source module and a power supply module electrically connected with the light source module, a heat dissipation module and a beam shaping module respectively connected with the light source module, and a light guide interface connected with the beam shaping module and aligned with a centre of the beam shaping module; the light source component is connected with the light guide component via the light guide interface; and the phototherapy component comprises a contactable photocosmetic device and/or a wearable phototherapy device; the power supply module is used for supplying power for the light source module, the light source module is used for emitting emergent light, the beam shaping module is used for performing beam shaping and contracting to the emergent light to improve light guiding efficiency, the light guide component is used for guiding the emergent light after the beam shaping and contracting to the phototherapy component and provide phototherapy to a user through the phototherapy component, and the heat dissipation module is used for exhausting thermal energy generated by the light source module, avoiding interference with the treatment that may be caused by thermal energy generated by the light source module of the light source component, which can further improve the treatment effect.

### BRIEF DESCRIPTION OF THE DRAWINGS

Other features, objects, and advantages of the present application will become more apparent by reading the detailed description of the non-limiting embodiments made with reference to the following drawings.

FIG. 1 shows a schematic diagram of modules of a phototherapy system according to an aspect of the present application;

FIG. 2 shows a schematic diagram of modules of a phototherapy system according to another aspect of the present application;

FIG. 3 shows a schematic structural view of a light source component in a phototherapy system according to an aspect of the present application;

FIG. 4 is a schematic structural view of a phototherapy component of a phototherapy knee brace according to an aspect of the present application;

FIG. 5 and FIG. 6 show schematic structural views of a phototherapy system for a phototherapy knee brace without a fixing buckle in an embodiment of the present application;

FIG. 7 shows a schematic structural view of a phototherapy system corresponding to a phototherapy cap in an embodiment of the present application;

FIG. 8 shows a schematic structural view of a phototherapy system corresponding to a phototherapy sock in an embodiment of the present application;

FIG. 9 shows a schematic structural view of a phototherapy system corresponding to a phototherapy glove in an embodiment of the present application;

FIG. 10 shows a schematic structural view of a phototherapy system corresponding to phototherapy pants in an embodiment of the present application;

FIG. 11 shows a schematic structural view of a phototherapy system corresponding to a phototherapy vest in an embodiment of the present application;

FIG. 12 is a schematic structural view of a light source module of a light source component in a phototherapy system according to an aspect of the present application;

FIG. 13 shows a schematic structural view of a beam shaping module of a tapered cylindrical shape in a phototherapy system according to an aspect of the present application;

FIG. 14 shows a schematic structural view of a beam shaping module of a circular cylindrical shape in a phototherapy system according to an aspect of the present application;

FIG. 15 shows a schematic structural view of a beam shaping module including a lens group in a phototherapy system according to an aspect of the present application;

FIG. 16 shows a schematic module diagram of a phototherapy system according to yet another aspect of the present application;

FIG. 17 shows a schematic diagram showing that the second optical fiber filaments in the optical fiber layer in the phototherapy system are arranged according to an aspect of the present application;

FIG. 18 is a schematic diagram showing that the second optical fiber filaments in the optical fiber layer in the phototherapy system are interwoven braids according to an aspect of the present application;

FIG. 19 is a schematic structural view of a phototherapy system according to an aspect of the present application;;

FIG. 20 shows a schematic structural view of a phototherapy system according to another aspect of the present application;

FIG. 21 shows a schematic structural view of a side-emitting fiber in an optical fiber layer in a phototherapy system according to another aspect of the present application;

### REFERNCE NUMERALS

1 Light source module, 2 phototherapy module, 3 light guide module, 11 light source module, 12 power supply module, 13 heat dissipation module, 14 beam shaping module, 15 light guide interface, 16 housing, 17 button, 21 wearable phototherapy device, 22 contactable photocosmetic device, 211 optical fiber light-emitting unit, 212 wearable unit, 23 fixation buckle, 2111 optical fiber filament, 2112 light emitting area, 21′ phototherapy cap, 21″ phototherapy socks, 21′″ phototherapy gloves, 21″″ phototherapy pants, 21′″″ phototherapy vest, 101 LED chip, 102 thermally conductive substrate, 103 power supply electrode, 104 mounting hole, 141 tapered light guide post, 141′ round light guide post, 142 plano-convex lens, 143 lens group, 221 optical fiber layer, 222 contact Layer, 223 reflective layer, 2211 second optical fiber filament, 22111 fiber core, 22112 cladding layer, 22113 side of side-light emitting optical fiber;

The same or similar reference numerals in the drawings represent the same or similar components.

### DETAILED DESCRIPTION

The present application will be described in further detail below with reference to the drawings.

As shown in FIG. 1, a phototherapy system in an embodiment of the present application includes a light source component 1 and a phototherapy component 2 (the phototherapy system may include N phototherapy components, for example, a phototherapy component V1, a phototherapy component V2, a phototherapy component V3, . . . , a phototherapy component VN shown in FIG. 1, where N is a positive integer greater than or equal to 1) and a light guide component 3 disposed between the light source component 1 and the phototherapy component 2 (wherein, each phototherapy component 2 in the contactable phototherapy system corresponds to a light guide component 3, for example, the light guide component ml in FIG. 1 corresponds to the phototherapy component V1, and the light guide component m2 corresponds to the phototherapy component V2, . . . and the light guide component mN corresponds to the phototherapy component VN), wherein the light guide component 3 conducts the light energy generated by the light source component 1 to the phototherapy component 2, and provides phototherapy to a user through the phototherapy component 2, and because the light guide component has a good electrical insulation and thermal insulation, therefore users can use the phototherapy system without risking electric leakage or excessive local temperature, thus the phototherapy system has high safety and no side effects.

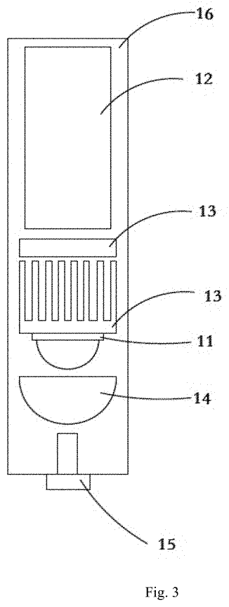

As shown in FIG. 3, the light source component 1 in the phototherapy system includes: a light source module 11, a power supply module 12, a heat dissipation module 13, a beam shaping module 14, a light guide interface 15 and a housing 16, wherein the housing 16 is made of metal or plastic; the power supply module 12 is electrically connected with the light source module 11 to provide power for the light source module 11.

The light source module 11 is connected with the heat dissipation module 13 and the beam shaping module 14 respectively, so that the heat dissipation module 13 discharges the thermal energy generated by the light source module 11 to avoid reducing the luminous efficiency due to overheating of the light source module and affecting therapeutic effect; the light source module 11 is used for emitting emergent light, which may include, but not limited to, spectrum such as ultraviolet, visible, and infrared light for therapeutic. Here, the light source module 11 in all the embodiments of the present application may include, but is not limited to, LED light source, laser light source, halogen light source and gas discharge light source, etc. The wavelength of the light emitted by the light source module 11 includes one or more of ultraviolet light, visible light or infrared light.

The beam shaping module 14 connected with the light source module 11 performs beam shaping and contracting on the emergent light emitted from the light source module 11, so that the light transmitted from the light guide component 3 to the optical fiber layer and used for treatment is emergent light having been subjected to beam shaping and contracting by the beam shaping module 14, so as to improve the guide efficiency of light energy from the light source module 11 to the light guide component 3.

The light guide interface 15 is connected with the beam shaping module 14 and has an interface for connecting the light guide component, and the light guide interface 15 is arranged to be aligned with the centre of the beam shaping module 14, to ensure that the emergent light can enter the light guide component 3 through the light guide interface, to realize the connection between the light source component 1 and the light guide component 3 through the light guide interface 15.

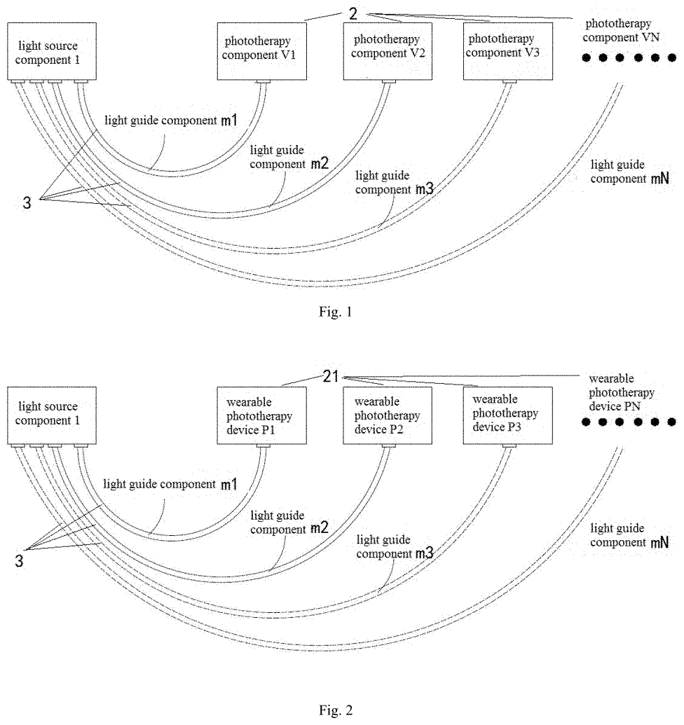

In the embodiment of the present application, the phototherapy component 2 may include either a wearable phototherapy device 21 as shown in FIG. 2 or a contactable photocosmetic device 22 as shown in FIG. 16, wherein:

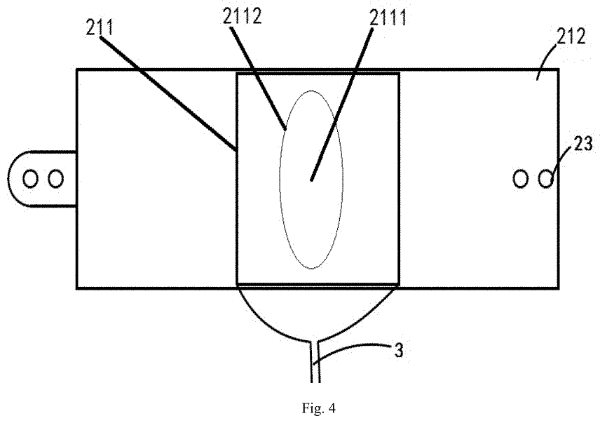

as shown in FIG. 4, the wearable phototherapy device 21 includes an optical fiber light-emitting unit 211 and a wearable unit 212, wherein the optical fiber light-emitting unit 211 has one end connected with the light guide component 3 and the other end connected with the wearable unit 212; the light guide component 3 conducts beam shaping and contracting to the emergent light generated in the light source module 11 in the light source component 1 through the beam shaping module 14, before guiding the light to the optical fiber light-emitting unit 211 to provide phototherapy for users wearing the wearable unit 212.

In an embodiment of the present application, the power supply module 12 may include: a control circuit and a direct constant current drive power supply, wherein the direct constant current drive power supply provides power to the light source module 11, and the control circuit is used for controlling changes in the power supplied to the light source module 11. In another embodiment, the power supply module 12 may include: a control circuit, a direct constant current drive power supply and a battery, so that the phototherapy system can be powered by the battery without direct connection with a power supply, thus the phototherapy system is potable, meeting the user's needs for the portability of the phototherapy system.

In this embodiment, since the LED light source and the power supply module are installed inside the light source component, during use, the optical fiber that is in contact with the surface of the treatment site (body surface, etc.) of the user-to-be-treated conducts light and not heat or electricity, there is no risk of electricity leakage or excessive local temperature, avoiding hidden safety hazards for the user during the use of phototherapy, rendering the use safer.

As shown in FIG. 12, the LED light source includes an LED chip 101, a thermally conductive substrate 102, a power supply electrode 103, and a mounting hole 104. The LED chip 101 is disposed at the centre of the thermally conductive substrate 102, and light emission by the LED chip is controlled by the power supply through the power supply electrode 103. The entire LED light source is installed on the light source component 1 through the mounting hole 104. There is one or more LED chips 101. The spectrum of the emergent light emitted by the LED light source in the light source component 1 is determined by the type and number of the LED chips 101.

In an embodiment of the present application, the heat dissipation module 13 may include a heat radiator, which can discharge the thermal energy generated by the light source module 11 in time, thereby ensuring the life and efficiency of the light source module 11; or, the heat dissipation module 13 may include a heat radiator and a fan which are combined to discharge the thermal energy generated by the light source module 11 timely and quickly, further ensuring the life and efficiency of the light source module 11.

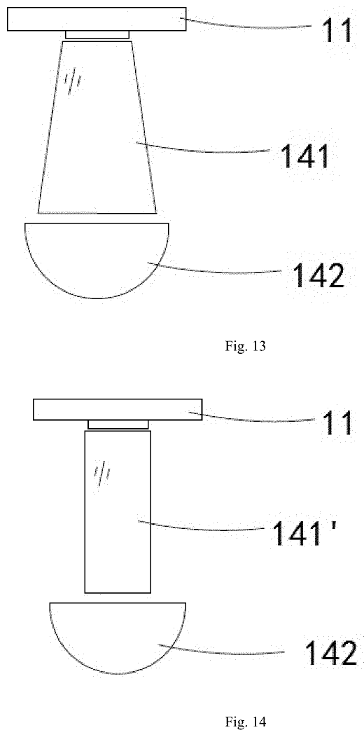

The beam shaping module 14 in the phototherapy system in an embodiment of the present application includes: a light guide post and a plano-convex lens 142, the light guide post is a tapered cylinder (as shown by the light guide post 141 in FIG. 13) or a circular cylinder (as shown by the circular light guide post 141′ in FIG. 14), the light guide post 141 is optically coupled to the light source module 11 and the plano-convex lens 142 respectively, centre lines of the light source module 11, the light guide post 141, and the plano-convex lens 142 are located on the same straight line. The light guide post 141 performs spectral mixing on the emergent light emitted from the light source module 11 to realize spectral mixing of the emergent light emitted from the light source module 11, to make the mixed spectrum more uniform; the plano-convex lens 142 is used for compressing the emergence angle of the emergent light after spectral mixing to obtain the emergent light after beam shaping and contraction to improve the optical coupling efficiency. The tapered structure of the tapered light guide post 141 in FIG. 13 can compress the emergence angle of the emergent light emitted by the light source module 11 to further improve the efficiency of light energy entering the light guide component.

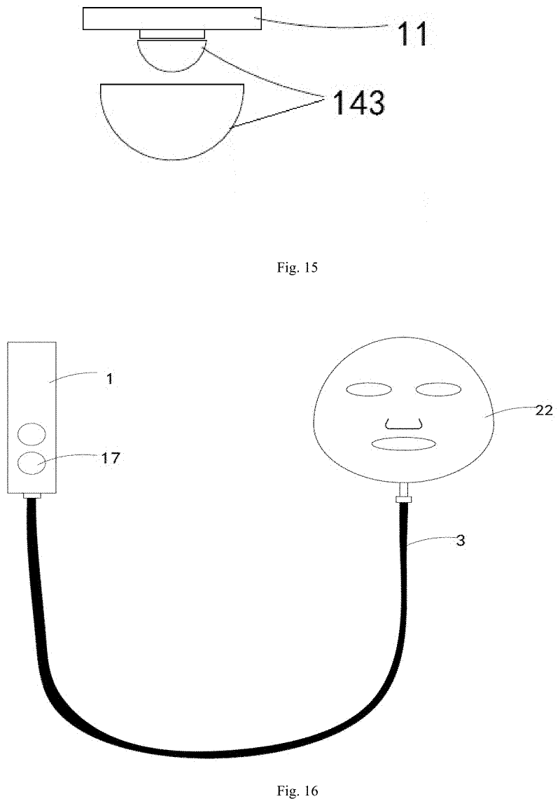

As shown in FIG. 15, the beam shaping module 14 in the phototherapy system in another embodiment of the present application includes: a lens group 143, which includes two plano-convex lenses and has one side optically coupled with the light source module 11 and the other side optically coupled with the light guide interface 15, and the lens group 143 is used for compressing the emergence angle of the emergent light emitted by the light source module 11.

In this embodiment, the light guide post and/or the lens group and the lens in the beam shaping module 14 are made of high-transmittance materials, so that the emergent light from the light source module 11 (such as an LED light source) is efficiently coupled into the light guide component 3, thereby improving the efficiency of the light energy being conducted.

Of course, the beam shaping module 14 in the phototherapy system of the present application may include not only the structure of the light guide post and the plano-convex lens, but also the structure of the lens group, and any other existing or future forms of structures of the beam shaping module 14 for performing beam shaping and contracting to the emergent light can be included in the protection scope of the present application.

As shown in FIG. 1, the phototherapy system in the embodiment of the present application includes: a light source component 1, one or more of the phototherapy components 2 and the light guide assemblies 3 corresponding thereto, and one light source component 1 can be simultaneously used in conjunction with multiple sets of the light guide components 3 and the phototherapy components 2 to improve the portability of the design; the light source component 1 is also provided with multiple light guide interfaces 16 to be connected with the light guide components 3 respectively; that is, there may be one or more phototherapy components 2 in the phototherapy system in the embodiments of the present application, and each phototherapy component 2 corresponds to a light guide component 3 and is connected with the light source component 1, which can realize simultaneous phototherapy for multiple parts of multiple users and improve practicality of the phototherapy system.

In conjunction with this embodiment, if the light source module 1 can connect up to N light guide modules and corresponding phototherapy modules, where N is a positive integer greater than or equal to 1, the interior of the light source module 1 includes: a power supply module 12, a heat dissipation module 13, N light source modules 11 and their corresponding N beam shaping modules 14 and N light guide interfaces 15, wherein the light source modules 11 of the N light source modules 11 are individually and simultaneously fixed to the surface of the radiator of the heat dissipation module 13, to be fixedly connected with the surface of the radiator, so that the thermal energy generated by each light source module can be discharged in time; and each light source module 11 corresponds to a group of the beam shaping modules and a group of light guide interfaces, to ensure that the emergent light emitted by each light source module 11 can pass through the corresponding beam shaping module 14, the light guide interface 15, and the light guide component 3 to conduct the corresponding emergent light after being shaped and contracted to the phototherapy component 2 correspondingly connected with the light guide component, to achieve the orientation of different parts of different users and effective phototherapy.

In the phototherapy system in an embodiment of the present application, the wearable phototherapy device 21 includes an optical fiber light-emitting unit 211 and a wearable unit 212, wherein the optical fiber light-emitting unit 211 is a whole piece of optical fiber fabric formed from the first optical fiber filaments woven together, wherein the material of the first optical fiber filament is plastic, and the wearable unit 212 is made of a skin-friendly material, so that the user can attach the wearable unit to the site where phototherapy treatment is needed, without hurting the skin at the treated site; through this light guide component 2, the emergent light from the light source module 11 can be conducted to the first optical fiber filament in the phototherapy assembly 2, and with the first optical fiber filaments bent during the weaving process, the emergent light generated by the light source module 11 leak from the side at the curved portion, so that the whole piece of optical fiber fabric forms a surface light source, which is attached with a treated part of the user in need of treatment through the wearable unit to perform phototherapy to the treated part. In this way, the emergent light forms a surface light source in the woven whole piece of optical fiber fabric.

In the phototherapy system of an embodiment of the present application, the two ends of the light guide component 3 are respectively provided with interfaces for connecting the light source component 1 and the wearable phototherapy device 21; during use of the interface, the light guide component the interface at one end of the light guide component 3 is connected with the corresponding light guide interface 16 in the light source component 1, and the interface at the other end of the light guide component 3 is connected with the interface at one end of the corresponding optical fiber light-emitting unit 211, to allow the emergent light emitted by the light source module in the light source component 1 to be transmitted to the optical fiber light-emitting unit 211 in the wearable phototherapy device 21 after beam shaping and contraction, so as to connect the light source component 1 with the wearable phototherapy device 21.

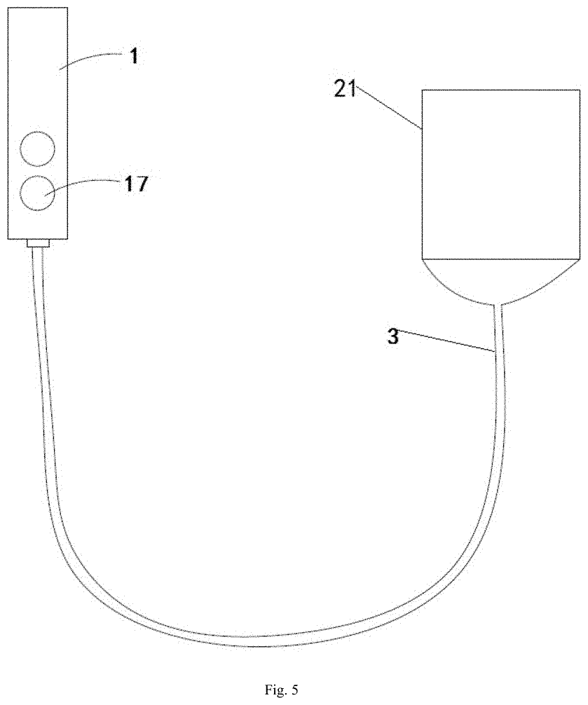

In the phototherapy system of an embodiment of the present application, the light source component 1 is provided with a button 17, as shown in FIGS. 4 and 5, the button 17 in the light source component 1 can be used to switch the wavelength and/or intensity of the emergent light, or to adjust the lighting mode, the phototherapy system can be adjusted to match different skins with different lighting schemes, and the different lighting schemes and the switching of the emergent light can be achieved by triggering the button 17 in the light source component 1, so as to meet the needs for different lighting by different users during the phototherapy.

In the phototherapy system in an embodiment of the present application, the phototherapy component 2 is provided with two sets of corresponding fixation buckles 23 as shown in FIG. 3, which are used to fix the treatment site of the user during the treatment to perform effective phototherapy to the treatment site as needed.

In the phototherapy system in all the embodiments of the present application, the form of the wearable phototherapy device 21 may have various forms, including at least any one of the following:

wearable hats, wearable scarfs, wearable vests, wearable aprons, wearable pants, wearable eye masks, wearable face masks, wearable elbow sleeves, wearable socks, wearable knee braces, wearable shoulder straps, wearable gloves and wearable belts. Of course, other forms of the wearable phototherapy device 21 applicable to the present application can be applied to the present application, and all belong to the protection scope of the present application.

As shown in FIG. 4, an embodiment of the present application provides a schematic structural view of a contactable phototherapy device 21 of a phototherapy knee brace, with a middle area thereof being a light-emitting area 2112 woven from a first optical fiber filament 2111. During use, the light-emitting area 2112 is attached with and wraps the treatment site to be treated, and fixed by fixing buckles 23 on both sides; the light emitted from the light-emitting area 2112 is introduced by the light guide component 3, and the incoming spectrum (emitted light) is determined by the emitted wavelength and intensity of the LED light source.

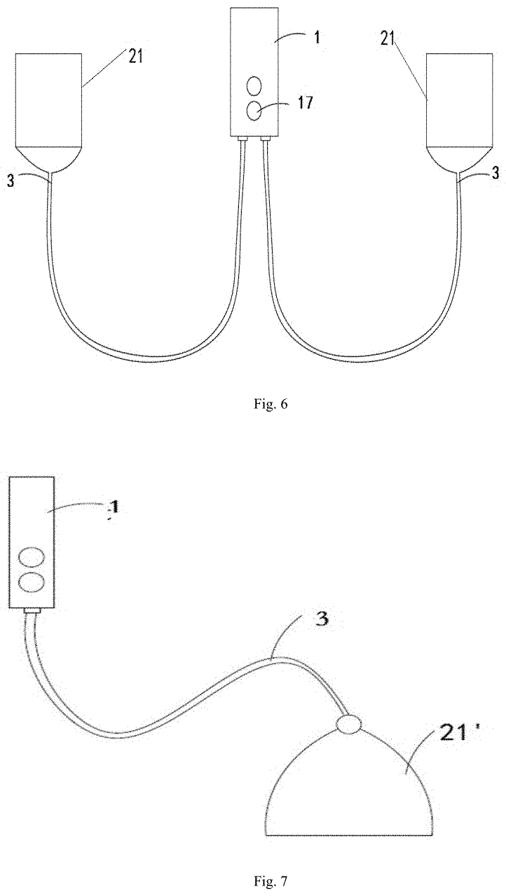

As shown in FIGS. 5 and 6, an embodiment of the present application provides a schematic diagram of an optical transmission structure of a phototherapy system of a phototherapy knee brace without a fixation buckle, wherein the optical fiber light-emitting unit 211 in the wearable phototherapy device 21 in FIG. 5 is composed of one piece of optical fiber cloth woven from the first optical fiber filaments, and the optical fiber light-emitting unit 211 in the wearable phototherapy device 21 in FIG. 6 is composed of two pieces of optical fiber fabric woven from the first optical fiber filaments, which can provide phototherapy to both knees simultaneously.

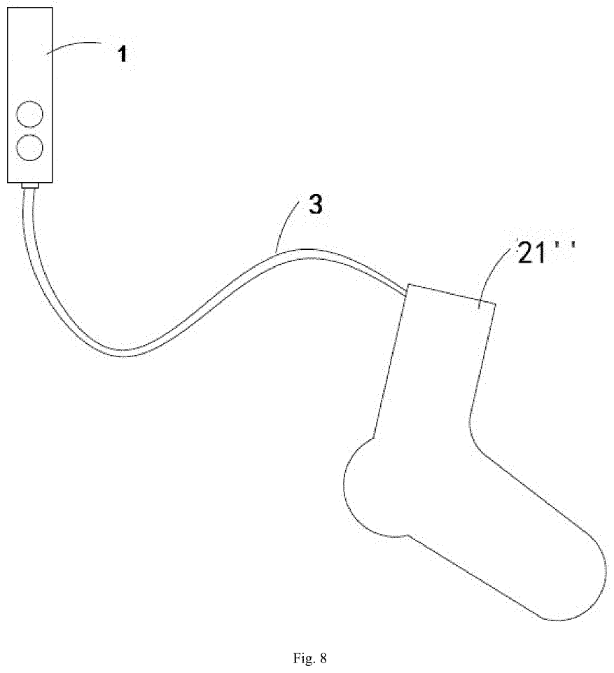

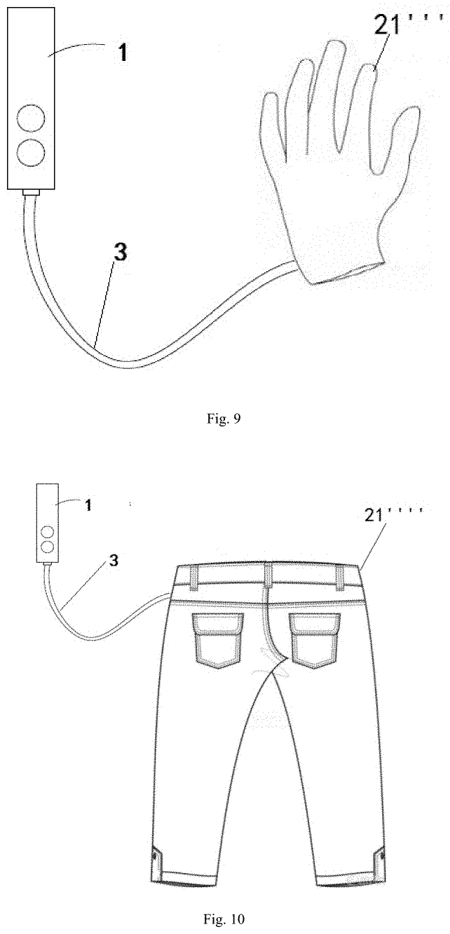

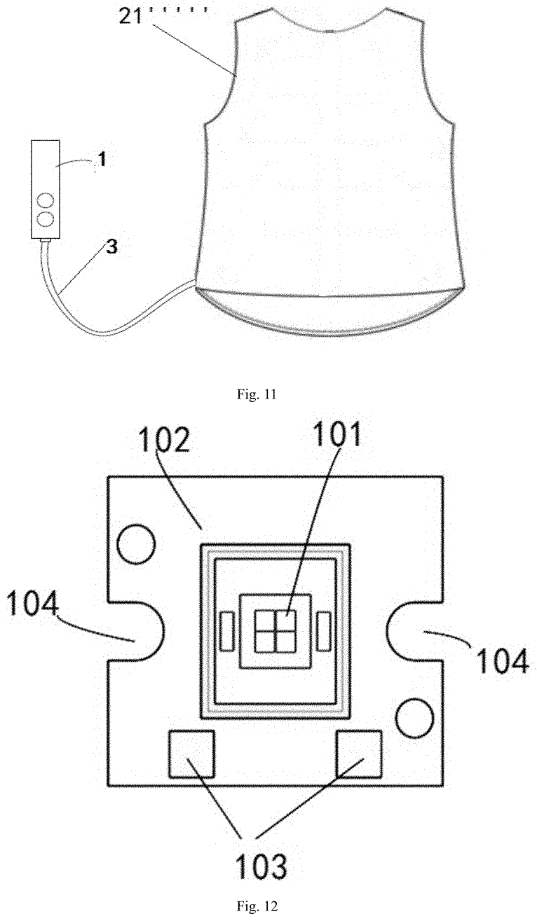

FIGS. 7 to 11 are respectively schematic structural views of a phototherapy system corresponding to a phototherapy cap, a phototherapy sock, a phototherapy glove, phototherapy pants and a phototherapy vest in an embodiment of the present application, wherein the light source component 1 of the phototherapy cap 21′ in FIG. 7 can be used to treat hair loss when it emits red light and near infrared light; the light source component 1 of the phototherapy socks 21 “in FIG. 8 irradiates with red light and blue light at the same time to achieve the effect of sterilization, inflammation and anti-swelling; the phototherapy gloves 21′” in FIG. 9 is used for the treatment of rheumatism in the hands of the user by the corresponding light emitted from the light source component 1; the phototherapy pants 21′ in FIG. 10 performs treatment to the thighs and calves of the lower body and body parts in contact with the pants of a user using the emergent light correspondingly emitted by the light source component 1, and can relieve the symptoms of lower body hemiplegia caused by rheumatism, etc.; the phototherapy vest 21′″″ in FIG. 11 can take care of or massage the user's back with the emergent light emitted from the phototherapy component 1, to realize effects of dampness removal and fatigue relief. The spectrum (emergent light) emitted from the light source component 1 of the present application for phototherapy can be adjusted according to user needs, so as to achieve specific targeted therapy. In addition, there are phototherapy components such as phototherapy belts and phototherapy shoulder straps, which cooperate with a light source component with corresponding wavelengths to realize phototherapy or health care for different parts of the user.

A phototherapy system provided by the present application includes a light source component, a light guide component, and a phototherapy component. When the phototherapy component is a wearable phototherapy device, the light source component can emit light of one or more specific wavelengths for illumination during phototherapy, the light source can be an LED light source; the light guide component is arranged between the light source component and the wearable phototherapy device, and is used to conduct the light energy generated by the light source component to the wearable phototherapy device. The wearable phototherapy system provided by the present application can use light to treat skin diseases, rheumatism and other diseases or for health care, and irradiate a specific spectrum of light uniformly on the surface to be treated via the side light-emitting optical fiber module. And because the optical fiber has good insulation, guides light, but does not conduct heat, the phototherapy system of the present application adopts the LED light source, and thus has the advantages of high safety and no side effects, and good market promotion value compared with the existing traditional equipment.

In another embodiment of the present application, when the phototherapy component 2 is a contactable photocosmetic device 22, as shown in FIG. 16, the contactable photocosmetic device 22 includes an optical fiber layer 221 which has second optical fiber filaments 2211 distributed therein and connected with the light guide component 3, and transmits the emergent light that has been shaped and contracted by the beam shaping module 14 to the optical fiber layer 221 through the light guide component 3, thereby providing phototherapy to users attached to the phototherapy system, so as to realize contact phototherapy to the corresponding skin part of the user who needs lighting hot compress, so as to achieve the purpose of beautifying the user's skin.

It should be noted that the materials of the second optical fiber filaments and the first optical fiber filaments in the embodiment of the present application are the same or belong to optical fiber filaments composed of the same or similar material, and the number is at least one for both the second optical fiber filaments and the first optical fiber filaments, that is, the first optical fiber filament is composed of one or more optical fibers, and the second optical fiber filament is also composed of one or more optical fibers.

The form of the contactable photocosmetic device 22 in the phototherapy system may include: contactable facial masks, contactable nasal masks, and contactable eye masks, etc.; of course, other forms of contactable photocosmetic devices 22 applicable to the present application can be applied to the present application, and are within the protection scope of the present application.

The light source module 11 in an embodiment of the present application is an LED light source as shown in FIG. 12, the LED light source can emit light of one or more wavelengths, and output specific light according to required spectrum and energy, and the light is transmitted to the second optical fiber filament 2211 in the optical fiber layer 221 of the contactable photocosmetic device 22 through the light guide component connected with the light guide interface 15. The material of the second optical fiber filament 2211 can be plastic, for performing illumination photo-cosmetology to contactable parts on the skin surface of the user.

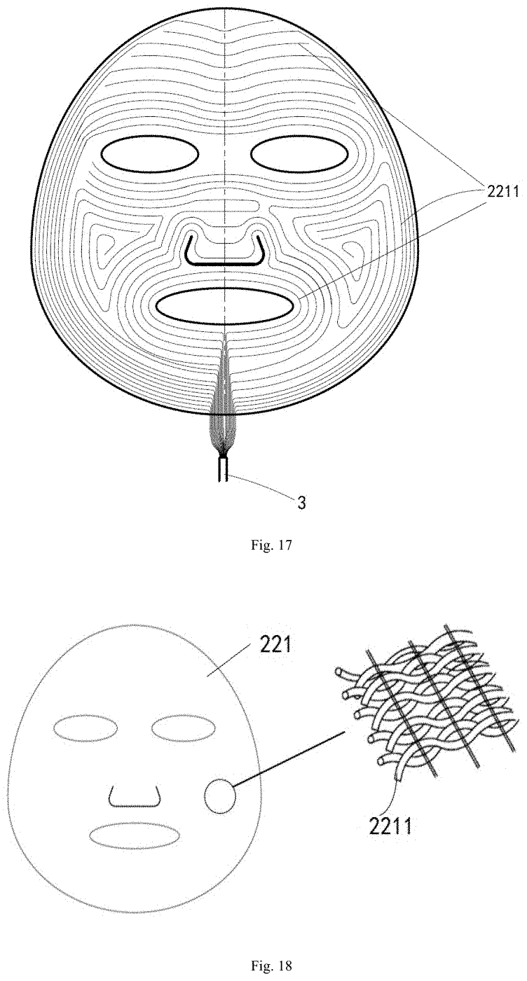

In the phototherapy system in an embodiment of the present application, the contactable photocosmetic device 22 includes an optical fiber layer 221, which can form a whole piece of optical fiber fabric from the second optical fiber filament 211 arranged in a certain layout, as shown in FIG. 17. Or the optical fiber layer 2211 forms a whole piece of optical fiber fabric from the second optical fiber filaments 2211 woven together and intersected in the optical fiber layer 221, as shown in FIG. 18. The light guide component 3 transmits the emergent light after the beam shaping and contraction to the second optical fiber filament 2211, allowing the emergent light to form a surface light source in the optical fiber fabric, and the surface light source engages with skin parts of the user in need of phototherapy through the optical fiber layer or the contact layer, to achieve the purpose of phototherapy.

In this embodiment, the contactable photocosmetic device 22 in the phototherapy system shown in FIG. 18 is composed of only the optical fiber layer 221, the second optical fiber filaments 2211 composed of side light-emitting optical fiber are interweaved in the optical fiber layer 221. As shown in FIG. 21 showing a schematic structural diagram of the side light-emitting optical fiber, the side light-emitting optical fiber is interwoven in the optical fiber layer. The side light-emitting optical fiber is made of transparent plastic, and the curved optical fiber emits light from the side thereof, thereby achieving uniform light emission of the optical fiber layer 221 in the phototherapy system. FIG. 18 is a schematic diagram of the actual application when the contactable photocosmetic device 22 in the phototherapy system of the present application is only an optical fiber layer, and the contactable photocosmetic device 22 is a contactable facial mask. In the phototherapy system corresponding to the contactable photocosmetic device 22 in FIG. 18, the emergent light is emitted by the light source module 11 in the light source component, passes through the beam shaping module 14 for beam shaping and contracting so that the emergence angle of the emergent light is compressed, realizing efficient coupling of light energy, then the emergent light after the beam shaping and contraction (emergent light with a more uniform spectrum) is transmitted through the light guide component 3 to the optical fiber layer 221 in the contactable photocosmetic device 22 to provide phototherapy to the user attached to the contactable photocosmetic device 22, so as to realize contact phototherapy to corresponding skin part of a user who needs light hot compress.

Of course, in the actual application schematic diagram of FIG. 18 in this embodiment, after the second optical fiber filaments 2211 are interwoven in the optical fiber layer 221 in a braided form, in order to improve the fitness between the contactable photocosmetic device 22 with the skin part of the user in need of the phototherapy and the user's experience, after the second optical fiber filaments 2211 are interwoven in the optical fiber layer, a highly active adsorbent material such as silicate gel is dissolved in the optical fiber layer 221 in which the second optical fiber filaments 2211 are interwoven, making the surface of the optical fiber layer 221 flatter and smoother, thereby improving the fitness with the skin part of the user requiring the phototherapy and the user's experience. And because the material of the second optical fiber filament 2211 in the optical fiber layer 221 is plastic which guides light but does not conduct heat or electricity, allowing the user to directly apply the optical fiber layer to the skin part that needs phototherapy, without causing any safety problem such as electricity leakage, and improve the user's safety.

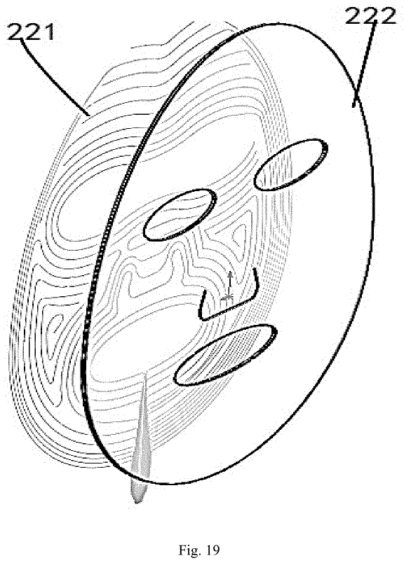

In an embodiment of the present application, as shown in FIG. 19, the contactable photocosmetic device 22 in the phototherapy system of the present application further includes a contact layer 222 attached with the optical fiber layer 221, namely, the contactable photocosmetic device 22 includes an optical fiber layer 221 and a contact layer 222 with similar overall sizes and shapes, to be fit and connected with each other, so that the optical fiber layer 221 is in contact with and attached to the skin part of a user needing phototherapy via the contact layer 222, the second optical fiber filaments 2211 distributed in the optical fiber layer 221 connected with the contact layer 222 emits light from the side thereof, so as to perform phototherapy for the user. Of course, when the contact layer 222 is in contact with the skin area that needs to be treated, a contactable film containing skin nutrient solutions, such as an existing facial mask, eye mask, and nasal mask, etc., can be used to provide skin care such as hydration, whitening and skin supplementation to the user's skin, during the phototherapy to the user's to be treated skin by the optical fiber layer 221, further realizing phototherapy and maintenance of the skin, and improving the user's experience of the phototherapy system.

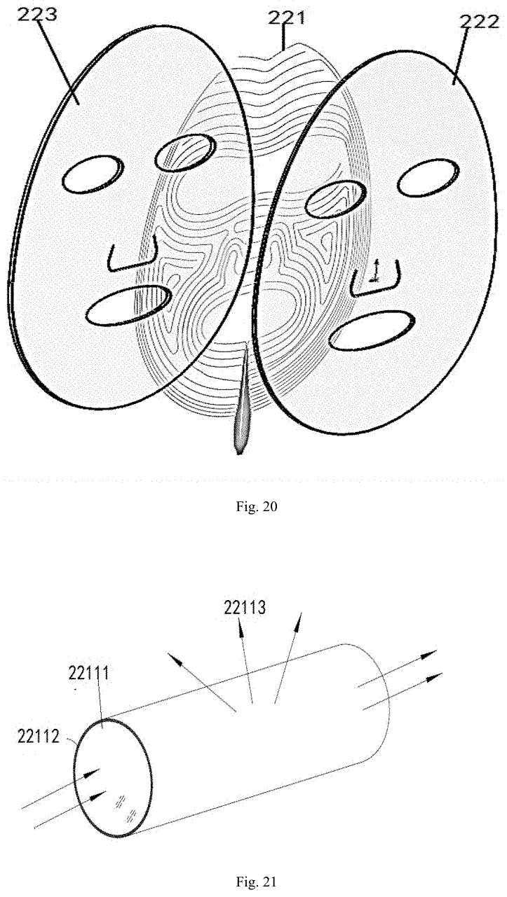

In another embodiment of the present application, as shown in FIG. 20, the contactable photocosmetic device 22 further includes a contact layer 222 and a reflective layer 223. The optical fiber layer 221 has one side attached with the contact layer 222, and the other side attached with the reflective layer 223, that is, the photocosmetic device 22 includes the optical fiber layer 221, the contact layer 222, and the reflective layer 223 with similar overall sizes and shapes so as to be fitted and connected with each other. The second optical fiber filament 2211 in the optical fiber layer 221 emits light from the side thereof, after side light emission by the optical fiber layer 221, although one side of the optical fiber layer 221 is attached with the contact layer 222, realizing phototherapy to the skin part of the user where needed, the other side of the fiber layer 221 can also emit light, causing waste and loss of light energy, hence the reflective layer 223 is provided on the other side of the fiber layer 221, and the light emitted on the other side is reflected by the reflective layer 223 back to the optical fiber layer 221, which not only avoids the energy waste of the emergent light transmitted to the contactable photocosmetic device 22, but also effectively improves the utilization rate of the emergent light transmitted to the optical fiber layer 221, maximizing the utilization rate of the emergent light.

In the above-mentioned embodiments of the present application, the contactable photocosmetic device 22 may be a contactable mask, a contactable nasal mask, a contactable eye mask, etc. When the contactable photocosmetic device 22 is a contactable facial mask, the second optical fiber filaments 2211 are arranged or interweaved more densely in the face area thereof, so that the face that needs phototherapy can be effectively illuminated; when the contactable photocosmetic device 22 is a contactable nasal mask, the second optical fiber filaments 2211 are arranged or interweaved more densely in the nose area thereof, so that the nose and its surrounding area can get targeted illumination, realizing photo rejuvenation of the skin of the nose (for example, to remove blackheads and acne, etc.); when the contactable photocosmetic device 22 is contactable eye mask, the second optical fiber filaments 2211 are arranged or interweaved more densely in the upper and lower regions of the eye and the area around the corners of the eye therein, the skin around the eyes, such as the crow's feet, eye bags and dark circles, can get targeted illumination, achieving photo rejuvenation of the skin around the eyes, so that the user has better facial (including around the nose and the eyes, etc.) appearance.

In a phototherapy system of an embodiment of the present application, an edge of the optical fiber layer 221 is provided with a docking interface, two ends of the light guide component 3 are respectively provided with an interface connected with the light source component 1 and the contactable photo-cosmetic device 22; the interface on one end of the light guide component 3 is connected with the light guide interface 15, the interface on the other end of the light guide component 3 is connected with the docking interface of the optical fiber layer 221, so that the light guide component 3 allows the emergent light emitted from the light source module 11 in the light source module 1, after beam shaping and contraction by the beam shaping module 14, to be transmitted to the optical fiber layer 221 in the contactable photocosmetic device 22 as shown, thereby realizing connection of the light source module 11 with the photocosmetic device 22.

Another aspect of the present application also provides a phototherapy system, including a light source component, a phototherapy component, and a light guide component disposed between the light source component and the phototherapy component, wherein,

the light source component includes: a light source module and a power supply module electrically connected with the light source module, a heat dissipation module connected with the light source module, and a light guide interface connected with the heat dissipation module, wherein the centre of the light guide interface is aligned with that of the light source module;

the light source component is connected with the light guide component through the light guide interface;

the phototherapy component includes a contactable photocosmetic device and/or a wearable phototherapy device;

wherein, the power supply module is used to supply power to the light source module, the light source module is used to emit emergent light, and the light guide component is used to transmit the emergent light to the phototherapy component and provide phototherapy to a user via the phototherapy component; the heat dissipation module is used to discharge the thermal energy generated by the light source module.

In summary, a phototherapy system provided by the present application includes a light source component, a phototherapy component, and a light guide component disposed between the light source component and the phototherapy component. The light guide component has good insulation, only guides light , but does not conduct heat, so that the phototherapy system supplies power with high safety and is not liable to produce side effects. The light source component includes: a light source module and a power supply module electrically connected with the light source module, a heat dissipation module and a beam shaping module respectively connected with the light source module, wherein the centre of the light guide interface aligns with that of the beam shaping module, and the light source component and the light guide component are connected through the light guide interface; the contactable photocosmetic device includes an optical fiber layer, the optical fiber layer has second optical fiber filaments distributed therein, and the optical fiber layer is connected with the light guide component. The power supply module is used for supplying power for the light source module, the light source module is used for emitting emergent light, the beam shaping module is used for performing beam shaping and contracting to the emergent light, to improve light guiding efficiency. The light guide component is used for guiding the emergent light after the beam shaping and contracting to the optical fiber light-emitting unit, to provide phototherapy to a user wearing the wearable unit, so as to provide phototherapy and hot compress on the user's skin; the heat dissipation module is used for exhausting thermal energy generated by the light source module, avoiding possible interference of the thermal energy generated by the light source module of the light source component with the treatment, thereby improving the treatment effect, so that the photocosmetic device of the present application (such as phototherapy facial masks, phototherapy eye masks and phototherapy nasal masks, etc.) has the advantages of rich functions, high safety, reliability and portability, low manufacturing cost, and a good market promotion prospect.

Obviously, those skilled in the art can make various modifications and variations to the present application without departing from the spirit and scope of the present application.

In this way, if these modifications and variations to the present application fall within the scope of the claims of the present application and equivalent technologies thereof, the present application is also intended to include these modifications and variations.

For a person skilled in the art, obviously, the present application is not limited to the details of the above exemplary embodiments, and the present application can be implemented in other specific forms without departing from the spirit or basic features of the present application. Therefore, regardless of the point of view, the embodiments should be regarded as exemplary and non-limiting, the scope of the present application is defined by the appended claims rather than the above description, and all changes fall within the meaning and scope of equivalent elements of the claims are therefore intended to be included in the present application. The reference numerals in the claims should not be considered as limiting the claims involved. In addition, it is clear that the word “comprise” does not exclude other units or steps, and the singular form does not exclude the plural form. Multiple units or devices defined in the device claims can also be implemented by one unit or device through software or hardware. Words such as first and second are used to indicate names, rather than any particular order.

What is claimed is:

1. A phototherapy system, comprising a light source component, a phototherapy component and a light guide component arranged between the light source component and the phototherapy component, wherein,
the light source component comprises: a light source module and a power supply module electrically connected thereto, a heat dissipation module and a beam shaping module respectively connected with the light source module, and a light guide interface connected with the beam shaping module and aligned with a centre of the beam shaping module;
the light source component is connected with the light guide component via the light guide interface; and
the phototherapy component comprises a contactable photocosmetic device and/or a wearable phototherapy device;
wherein, the power supply module is used for supplying power for the light source module, the light source module is used for emitting emergent light, the beam shaping module is used for performing beam shaping and contracting to the emergent light, the light guide component is used for guiding the emergent light after the beam shaping and contracting to the phototherapy component and provide phototherapy to a user through the phototherapy component, and the heat dissipation module is used for exhausting thermal energy generated by the light source module.

2. The phototherapy system of claim 1, wherein, the wearable phototherapy device comprises: an optical fiber light-emitting unit and a wearable unit,
wherein, the optical fiber light-emitting unit has one end connected with the light guide component, and the other end connected with the wearable unit; and
the light guide component is used for guiding the emergent light after the beam shaping and contracting to the optical fiber light-emitting unit, to provide phototherapy to a user wearing the wearable unit.

3. The phototherapy system of claim 2, wherein, the optical fiber light-emitting unit is a whole piece of optical fiber fabric formed from a first optical fiber filament woven together,
wherein, the light guide component transmit the emergent light to the first optical fiber filament, to allow the emergent light in the whole woven piece of optical fiber fabric to form a surface light source.

4. The phototherapy system of claim 1, wherein, the phototherapy system comprises a light source component, one or more phototherapy components and the light guide component corresponding thereto.

5. The phototherapy system of claim 4, wherein, the light source component comprises: a power supply module, a heat dissipation module, one or more light source modules and the beam shaping module and the light guide interface corresponding thereto, wherein,
each light source module corresponds to a group of the beam shaping module and a group of the light guide interface.

6. The phototherapy system of claim 2, wherein, two ends of the light guide component are respectively provided with an interface connected with the light source component and the wearable phototherapy device; wherein,
the interface on one end of the light guide component is connected with the light guide interface, and
the interface on the other end of the light guide component is connected with an interface on one end of the optical fiber light-emitting unit.

7. The phototherapy system of claim 1, wherein, the contactable photocosmetic device comprises an optical fiber layer, having a second optical fiber filament distributed therein and connected with the light guide component;
wherein, the light guide component is used for guiding the emergent light after the beam shaping and contracting to the optical fiber layer, to provide phototherapy to a user attached to the contactable photocosmetic device.

8. The phototherapy system of claim 7, wherein, the optical fiber layer forms a whole piece of optical fiber fabric in the form of the second optical fiber filament in a certain layout, or the optical fiber layer forms a whole piece of optical fiber fabric in the form of the second optical fiber filament which is interwoven in the optical fiber layer and is woven together;
wherein, the light guide component transmits the emergent light after the beam shaping and contracting to the second optical fiber filament, to allow the emergent light in the optical fiber fabric after the beam shaping and contracting, to form a surface light source.

9. The phototherapy system of claim 8, wherein, the contactable photocosmetic device further comprises a contact layer, which is fitted and connected with the optical fiber layer; or the contactable photocosmetic device further comprises a contact layer and a reflective layer, wherein, the optical fiber layer has one surface fitted and connected with the contact layer, and another surface fitted and connected with the reflective layer.

10. The phototherapy system of claim 7, wherein, an edge of the optical fiber layer is provided with a docking interface, two ends of the light guide component are respectively provided with an interface connected with the light source component and the contactable photocosmetic device, wherein, the interface on one end of the light guide component is connected with the light guide interface, the interface on the other end of the light guide component is connected with the docking interface of the optical fiber layer; or
the edge of the optical fiber layer is provided with a docking interface, and only one end of the light guide component is provided with an interface fitted and connected with the docking interface, wherein, the docking interface secures the second optical fiber filament into a bundle of optical fiber via a hoop to form the interface of the light guide component.

11. The phototherapy system of claim 1, wherein, the beam shaping module comprises: a light guide post and a plano convex lens, wherein, the light guide post is respectively optically coupled with the light source module and the plano convex lens, and central lines of the light source module, the light guide post and the plano convex lens are along the same line, the light guide post is used for performing spectral mixing to the emergent light emitted by the light source module, the plano convex lens is used for compressing an emergence angle of the emergent light after the spectral mixing, to obtain emergent light after the beam shaping and contracting;
or,
the beam shaping module comprises a lens group, with one side thereof being in optical coupling connection with the light source module, and the other side thereof being in optical connection with the light guide interface, and being used for compressing the emergence angle of the emergent light emitted from the light source module.

11. The phototherapy system of claim 1, wherein, the heat dissipation module comprises a radiator, or a radiator and a fan,
wherein, the light source module is fixedly connected with a surface of the radiator of the heat dissipation module.

12. The phototherapy system of claim 1, wherein, the power supply module comprises: a control circuit and a direct constant current drive power supply; or
the power supply module comprises: a control circuit, a direct constant current drive power supply and a battery.

13. The phototherapy system of claim 1, wherein, the light source component is provided with a button, for switching wavelength and/or intensity of the emergent light, or adjusting a lighting mode.

14. The phototherapy system of claim 1, wherein, the phototherapy component is provided with two sets of corresponding fixation buckles, for fixing a treated site of the user during treatment process.

15. The phototherapy system of claim 1, wherein, the wearable phototherapy device is in at least any one of the following forms of:
wearable hats, wearable collars, wearable vests, wearable bellybands, wearable pants, wearable eye masks, wearable facial masks, wearable elbow protectors, wearable socks, wearable knee pads, wearable shoulder straps, wearable gloves and wearable belts;
the contactable photocosmetic device is in at least any one of the following forms of:
contactable facial masks, contactable nasal masks and contactable eye masks.

16. A phototherapy system, comprising a light source component, a phototherapy component and a light guide component provided between the light source component and the phototherapy component, wherein,
the light source component comprises: a light source module and a power supply module electrically connected therewith, a heat dissipation module connected with the light source module, a light guide interface connected with the heat dissipation module and having a centre aligned with that of the light source module;
the light source component is connected with the light guide component through the light guide interface;
the phototherapy component comprises a contactable photocosmetic device and/or a wearable phototherapy device;
wherein, the power supply module is used for supplying power for the light source module, the light source module is used for emitting emergent light, the light guide component is used for guiding the emergent light to the phototherapy component and provide phototherapy to a user through the phototherapy component, and the heat dissipation module is used for exhausting thermal energy generated by the light source module.
